(19) United States
(12) Patent Application Publication
(10) Pub. No.: US 2025/0073117 A1
(43) Pub. Date: Mar. 6, 2025

(54) THERAPEUTIC DEVICE AND METHOD OF MANUFACTURING THEREOF

(71) Applicant: SHENZHEN KAIYAN MEDICAL EQUIPMENT CO., LTD., Shenzhen (CN)

(72) Inventors: ALAIN DIJKSTRA, Amstelveen (NL); YUAN HUA DONG, Shenzhen (CN); LI XIANG, Shenzhen (CN)

(73) Assignee: SHENZHEN KAIYAN MEDICAL EQUIPMENT CO., LTD, Shenzhen (CN)

(21) Appl. No.: 18/408,626

(22) Filed: Jan. 10, 2024

(30) Foreign Application Priority Data
Sep. 5, 2023 (CN) 2023223971264

(51) Int. Cl. A61H 23/00; A61F 7/00; A61N 5/067

(52) U.S. Cl. CPC A61H 23/00; A61F 7/007; A61N 5/067; A61F 2007/0071; A61F 2007/0096; A61F 2007/0098; A61H 2201/013; A61H 2201/10; A61H 2201/5007; A61H 2201/5028; A61H 2201/5071; A61N 2005/0652; A61N 2005/0659; A61N 2005/0661

(57) ABSTRACT

Embodiments of the present invention provide a therapeutic device and a method of manufacturing thereof. The therapeutic device includes a first structure and a second structure connected through one or more first compressible elements. Furthermore, one or both of the first structure and the second structure include one or more stimulation elements. Also, the one or more first compressible elements are configured to transition between a compressed state and an expanded state.

### TECHNICAL FIELD

The present invention relates generally to recreational and therapeutic devices. More specifically, the present invention relates to therapeutic devices that use combinational therapies to provide alternate therapies for maladies such as acute or chronic pain, skin degeneration, and mental disorders such as anxiety, depression, post-traumatic stress, and cognitive decline.

### BACKGROUND ART

Combinational alternative therapies involving combinations of light therapy, heating, cooling, vibratory massage, electrotherapy, etc. have been used for quite some time to provide benefits such as pain relief, skin rejuvenation, wound healing, and improvement of overall mental health. However, such combinational therapies require the use of several distinct devices in a professional setup such as a spa, a gymnasium, or a salon. Alternatively, some apparatuses can combine several different therapies into a single machine. However, such solutions are generally inaccessible on a relatively wider scale owing to their bulk, design complexities, and prohibitive costs. Also, such apparatuses are power intensive and therefore lead to even higher operational costs downstream. As a result, there is a demand in the industry for a solution that overcomes the aforementioned deficiencies.

### OBJECTS OF THE INVENTION

Some of the objects of the invention are as follows:

An object of the present invention is to provide a therapeutic device that has a relatively very small footprint in terms of size and power consumption.

Another object of the present invention is to provide a therapeutic device that is capable of providing a combination of distinct therapies such as light therapy, heating, cooling, vibratory massage, electrotherapy, etc.

Another object of the present invention is to provide a therapeutic device that is convenient to hold during usage of the device and application of several different therapies.

Another object of the present invention is to provide a therapeutic device that can be operated remotely using a user computing device.

It is also an object of the present invention to provide a therapeutic device that can be carried easily through attachment with another device or another article.

### SUMMARY OF THE INVENTION

According to a first aspect of the present invention, there is provided a therapeutic device. The therapeutic device includes a first structure and a second structure connected through one or more first compressible elements. Furthermore, one or both of the first structure and the second structure include one or more stimulation elements. Also, the one or more first compressible elements are configured to transition between a compressed state and an expanded state.

In one embodiment of the invention, the one or more stimulation elements are selected from a group consisting of Light Emitting Diodes (LEDs), lasers, heating elements, cooling elements, vibration elements, electrodes, and combinations thereof.

In one embodiment of the invention, the first structure includes a supporting bracket, a Printed Circuit Board (PCB) located on a first side of the supporting bracket, and a first stimulation element, a plurality of second stimulation elements, and an electrical power source electrically coupled to the PCB, the first stimulation element and the plurality of second stimulation elements at least partially constituting the one or more stimulation elements. Further, the second structure includes a lid located on a second side of the supporting bracket, the second side being opposite to the first side. Also, the one or more first compressible elements are disposed between the supporting bracket surface and the lid, on the second side of the supporting bracket.

In one embodiment of the invention, the therapeutic device further includes one or more pressure sensors electrically coupled to the PCB. A second compressible element is in contact the one or more pressure sensors towards the second side. Moreover, the one or more pressure sensors are configured to sense a pressure exerted on the second compressible element for activation and deactivation of the one or more of the first stimulation element and the plurality of second stimulation elements.

In one embodiment of the invention, the first stimulation element is a heating plate located over the second compressible element, the heating plate thermally coupled to a resistive heating coil, one or more temperature sensors thermally coupled to the first stimulation element, the one more temperature sensors configured to sense a temperature of the first stimulation element, such that, in use, electrical power being supplied to the heating element is modified in correlation with the sensed temperature.

In one embodiment of the invention, in construction, the plurality of second stimulation elements defines a periphery surrounding the first stimulation element.

In one embodiment of the invention, the plurality of second elements includes Light Emitting Diodes (LEDs) and/or lasers, and the first structure further includes a diaphanous cover layer disposed over the plurality of second stimulation elements, towards the first side.

In one embodiment of the invention, the therapeutic device further includes an expansion switch coupled to the one or more first compressible elements, the expansion switch configured to activate the one or more stimulation elements when at least one of the one or more first compressible elements are in the expanded state and deactivate the one or more stimulation elements when each one of the one or more compressible elements are in the compressed state.

In one embodiment of the invention, the one or more stimulation elements include a plurality of irradiation sources configured to emit electromagnetic radiation with wavelength ranges of 300 nm to 1200 nm.

In one embodiment of the invention, at least one of the one or more first compressible elements includes an air transfer port configured to receive air when a respective first compressible element, of the one or more first compressible elements, transitions from the compressed state to the expanded state, and to release air when the respective first compressible element transitions from the expanded state to the compressed state.

In one embodiment of the invention, the therapeutic device further includes a receiver induction coil electrically coupled to the electrical power source, the receiver induction coil configured to receive electrical power from a time-varying magnetic field generated by a transmitter induction coil of a wireless charging device.

In one embodiment of the invention, the therapeutic device further includes a communication interface configured to receive a control input signal from a user computing device, such that, in use, operational characteristics of one or more of the first stimulation element and the plurality of second stimulation elements are modified in response to the receipt of the control input signal.

In one embodiment of the invention, one or both of the first structure and the second structure include a respective fastening arrangement configured to mechanically couple the first structure or the second structure to another object.

According to a second aspect of the present invention, there is provided a therapeutic device. The therapeutic device includes a first structure and a second structure connected through one or more first compressible elements, one or both of the first structure and the second structure including one or more stimulation elements. Furthermore, the one or more first compressible elements are configured to transition between a compressed state and an expanded state. Furthermore, the one or more stimulation elements are at least partially constituted by a first stimulation element and a plurality of second stimulation elements. Also, in construction, the plurality of second stimulation elements defines a periphery surrounding the first stimulation element.

In one embodiment of the invention, the first structure includes a supporting bracket, a Printed Circuit Board (PCB) located on a first side of the supporting bracket, the plurality of second stimulation elements, the first stimulation element, and an electrical power source electrically coupled to the PCB. Also, the second structure includes a lid located on a second side of the supporting bracket, the second side being opposite to the first side. Also, the one or more first compressible elements are disposed between the supporting bracket surface and the lid, on the second side of the supporting bracket.

In one embodiment of the invention, the therapeutic device further includes one or more pressure sensors electrically coupled to the PCB, a second compressible element in contact the one or more pressure sensors towards the second side. Also, the one or more pressure sensors are configured to sense a pressure exerted on the second compressible element for activation and deactivation of the one or more of the first stimulation element and the plurality of second stimulation elements.

In one embodiment of the invention, the first stimulation element is a heating plate located over the second compressible element, the heating plate thermally coupled to a resistive heating coil, one or more temperature sensors thermally coupled to the first stimulation element, the one more temperature sensors configured to sense a temperature of the first stimulation element, such that, in use, electrical power being supplied to the heating element is modified in correlation with the sensed temperature.

In one embodiment of the invention, the plurality of second elements comprises Light Emitting Diodes (LEDs) and/or lasers, and the first structure further includes a diaphanous cover layer disposed over the plurality of second stimulation elements, towards the first side.

In one embodiment of the invention, the therapeutic device further includes an expansion switch coupled to the one or more first compressible elements, the expansion switch configured to activate the one or more stimulation elements when at least one of the one or more first compressible elements are in the expanded state and deactivate the one or more stimulation elements when each one of the one or more compressible elements are in the compressed state.

According to a third aspect of the present invention, there is provided a method of manufacturing a therapeutic device. The method includes providing a first structure, a second structure, and one or more first compressible elements, connecting the first structure and the second structure through the one or more first compressible elements, and providing one or more stimulation elements within one or both of the first structure and the second structure. Furthermore, the one or more first compressible elements are configured to transition between a compressed state and an expanded state.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of the specification, the phrase “communication network” refers to a group of several connected devices including computing devices (such as desktops, mobile handheld devices, tablet PCs, notebooks, etc.), local and remotely located servers (such as web servers, application servers, database servers, Application Program Interface (API) servers, load balancers, compute nodes, and the like), routers, antennas, modems, multiplexers, demultiplexers, and the like. In that regard, the aforementioned connected devices may be able to exchange data signals through wired and/or wireless means as per several combinations of several different communication protocols such as 802.11 (Wi-Fi), 802.3 (Ethernet), Bluetooth, NFC, ZigBee and 3GPP protocols such as HSPA, HSDPA, LTE, GSM, CDMA, WLL and the like.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, a “diaphanous material” is a material that allows at least a portion of one or more forms (such as Infrared, Ultraviolet, X-Rays, Visible Light, Microwaves, Radio Waves, etc.) of electromagnetic radiation to pass through them. The diaphanous materials can be transparent (allowing the one or more forms of the electromagnetic radiation to pass through with minimal scattering) or translucent (allowing the one or more forms of the electromagnetic radiation to pass through with appreciable diffusion or scattering). Diaphanous materials can be dense, like glass, or have an open structure, like wire mesh or a woven fabric.

In the context of the specification, the term “historical” in execution of a command refers to anything pertaining to a time instant(s) that is earlier than a time instant of an initiation of the command.

In the context of the specification, the term, “real-time”, refers to without intentional delay, given the processing limitations of hardware/software/firmware involved and the time required to accurately measure/receive/process/transmit data as practically possible.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a top perspective view of a therapeutic device, in accordance with an embodiment of the present invention;

FIG. 1B illustrates a bottom perspective view of the therapeutic device of FIG. 1A;

FIG. 2 illustrates an exploded view of the therapeutic device, in accordance with an embodiment of the present invention;

FIG. 3A illustrates a front view of the therapeutic device in an open condition, in accordance with an embodiment of the present invention;

FIG. 3B illustrates a bottom view of the therapeutic device of FIG. 3A;

FIG. 3C illustrates a front sectional view of the therapeutic device of FIGS. 3A and 3B, sectioned along plane A-A of FIG. 3B;

FIG. 4A illustrates a front view of the therapeutic device in a closed condition, in accordance with an embodiment of the present invention;

FIG. 4B illustrates a bottom view of the therapeutic device of FIG. 4A;

FIG. 4C illustrates a front sectional view of the therapeutic device of FIGS. 4A and 4B, sectioned along plane B-B of FIG. 4B;

FIG. 5 illustrates the therapeutic device in communication with a user computing device, in accordance with an embodiment of the present invention;

FIG. 6 illustrates the therapeutic device mechanically coupled to another electronic device, in accordance with an embodiment of the present invention; and

FIG. 7 illustrates a flowchart depicting a method of manufacturing a therapeutic device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a therapeutic device. The therapeutic device includes two structures connected through compressible elements such as springs, elastic bellows, elastic chambers, etc. One or both of the two structures may include stimulation elements such as Light Emitting Diodes (LEDs), lasers, heating elements, cooling elements, vibratory elements, electrodes, etc. For example, one of the two structures may be provided with a resistive heating plate surrounded by several LEDs for providing a combinational therapy of heating and irradiation. Additional stimulation elements may also be provided on the second structure for a two-sided application of the combinational therapies. The compressible elements allow the therapeutic device to collapse for convenient and space-efficient storage.

Several additional functionalities may also be added to the therapeutic device. For example, an expansion switch may activate the therapeutic device when the compressible elements are expanded before the device is put to use. Moreover, activation and control of specific stimulation elements may be based upon feedback received from several sensors such as pressure sensors (for ascertaining contact with a body portion of the user) and temperature sensors (for maintaining the temperature of heating and/or cooling elements within permissible limits). Electrical power to operate the therapeutic device may be provided through an onboard rechargeable battery that may be charged using a power cable. The therapeutic device can also be controlled by a user computing device communicating with the therapeutic device, over a communication network, through a communication interface built into the therapeutic device. In addition, fastening arrangements may be provided on one or both the structures allowing the therapeutic device to be fastened to another object or another article during transportation.

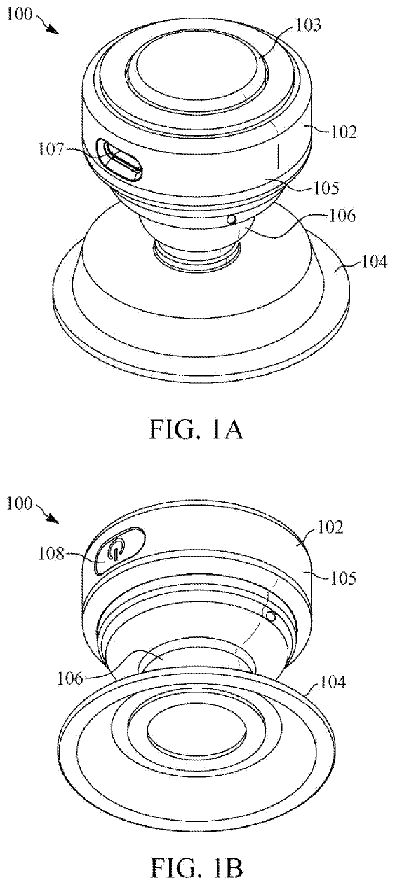

Several embodiments of the present invention will now be described in detail with references to FIGS. 1A-7.

FIG. 1A illustrates a top perspective view of a therapeutic device 100 (hereinafter also referred to as “the device 100”), in accordance with an embodiment of the present invention. FIG. 1B illustrates a bottom perspective view of the therapeutic device 100 of FIG. 1A. The device 100 includes a first structure 102 and a second structure 104 connected through one or more first compressing elements 106. The first structure 102 includes a housing structure 105 that encapsulates several different elements of the first structure 102. The one or more first compressing elements 106 are configured to transition between a compressed state (where the respective lengths of the one or more first compressing elements 106 are smaller than the respective maximum possible lengths) and an expanded state (where the respective lengths of the one or more first compressing elements 106 are equal to the respective maximum possible lengths). In that regard, the one or more first compressing elements 106 may include helical springs, bellows, fluid-filled chambers (with movable pistons, and intake and exhaust valves), etc. The compression of the one or more first compressing elements 106 allows the second structure 104 to be brought relatively closer to the first structure 102, thereby reducing the overall size of the device 100 and enabling easier and space-efficient storage and handling of the device 100. In FIGS. 1A and 1B, the one or more first compressing elements 106 have been depicted in the expanded state. Furthermore, the one or more first compressing elements 106 have been depicted to have a conical shape. The conical shape of the one or more first compressing elements 106 allows the device 100 to be held firmly in the hands of a user during usage.

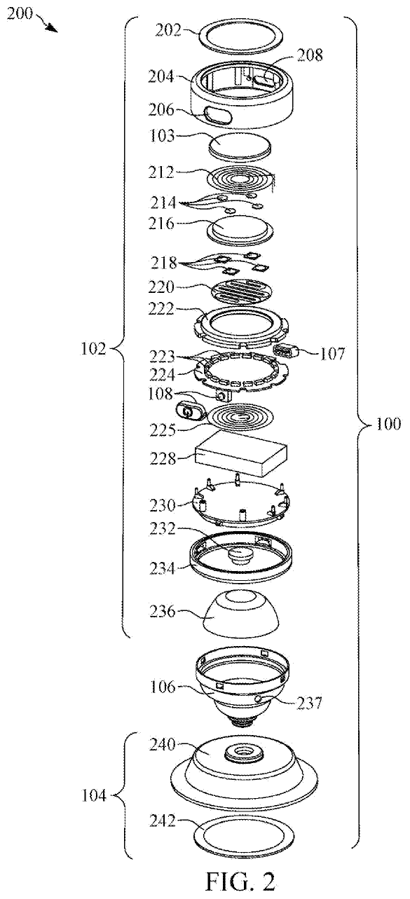

The device 100 further includes one or more stimulation elements (See FIG. 2) distributed amongst one or both of the first structure 102 and the second structure 104. In several embodiments of the invention, the one or more stimulation elements may be selected from a group consisting of Light Emitting Diodes (LEDs), lasers, heating elements, cooling elements, vibration elements, electrodes and combinations thereof. For example, FIG. 1A illustrates a first stimulation element 103 protruding above the surface of the first structure 102. The first stimulation element 103 may be an LED mesh, a laser, a heating element, a cooling element, a vibration element, an electrode, and combinations of any two or more of the aforementioned stimulation elements.

In the case of the first stimulation element 103 being the LED or the laser, the first stimulation element 103 may be emitting electromagnetic radiation. In the case of the first stimulation element 103 being the vibration element, the first stimulation element 103 may be a vibrating head connected to an eccentric mass rotating motor or a linear resonant motor. In the case of the first stimulation element 103 being the heating element, the first stimulation element 103 may be selected from a group consisting of metal heating elements, ceramic heating elements, semiconductor heating elements, thick film heating elements, polymer-based heating elements, composite heating elements, and combination heating elements. In the case of the first stimulation element 103 being the cooling element, the first stimulation element 103 may be a thermoelectric cooler, also known as a Peltier heat pump. In the case of the first stimulation element 103 being an electrode, the first stimulation element 103 may be embodied as an open-ended conductor. The first stimulation element 103 may then be able to provide Transcutaneous Electrical Nerve Stimulation (TENS), Electronic Muscle Stimulation (EMS), and Microcurrent Electrical Therapy (MET) to the body of a user.

TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

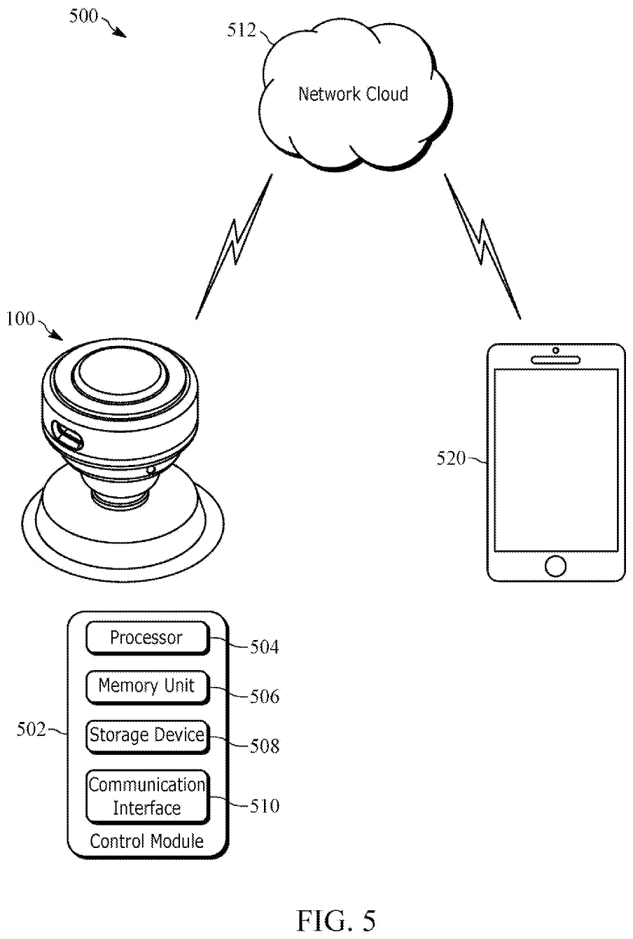

FIGS. 1A and 1B also illustrate a data and power transfer port 107. The data and power transfer port 107 may be configured to exchange data between the device 100 and another electronic device such as a user computing device (See FIG. 5). Furthermore, the data and power transfer port 107 may also be configured to receive or deliver electrical power, from and to, respectively, an external power source such as a power brick connected to AC power supply or a power bank having relatively larger battery capacity. In that regard, the data and power transfer port 107 may be selected from a group consisting of USB A/B/C ports, a micro-USB port, a lightening connector port, an HDMI port, an Ethernet port, etc. Furthermore, FIGS. 1A and 1B also illustrate a user interface 108. The user interface 108 may be embodied as a power on/off button, an array of buttons with mode selection options, an LED or LCD touch screen with resistive or capacitive touch sensors, etc. The touch screen in that regard may be configured to provide several alternate options such as mode selection, selection of types of therapy (such as irradiation, heating, cooling, etc.), selection of operational parameters (such as pulse width, intensity, wavelength of irradiation, maximum and minimum temperature in heating and cooling, frequency and amplitude in case of vibration therapy, and magnitude of current in case electrode based therapies).

FIG. 2 illustrates an exploded view 200 of the therapeutic device 100, in accordance with an embodiment of the present invention. FIG. 2 illustrates a first fastening arrangement 202 as a part of the first structure 102. The first fastening arrangement 202 is configured to mechanically couple the first structure 102 to another object. For example, the other object may be an electronic device such as a cellular phone, a Personal Digital Assistant (PDA), or the skin of the user. In that regard, the first fastening arrangement 202 may be a ferromagnetic strip, a polymeric, or a fabric strip provided with an adhesive agent. The first structure 102 further includes a first housing segment 204 and a second housing segment 234. The first housing segment 204 and the second housing segment 234 together combine to form the housing structure 105. The first housing segment 204 has been provided with a user interface cutout 206 and a data and power transfer port cutout 208. The user interface cutout 206 enables the location of the user interface 108 with the housing structure 105, and the data and power transfer port cutout 208 enables the location of the data and power transfer port 107 with the housing structure 105.

The first structure 102 further includes the first stimulation element 103. The first stimulation element 103 may be directly activated or deactivated through the user interface 108. Moreover, in several embodiments, several modes of operations of the first stimulation element 103 may be set or activated using the user interface 108. In the given embodiment, the first stimulation element 103 is a heating plate thermally coupled to a resistive heating coil 212. In several embodiments of the invention, one or more temperature sensors 214 may also be coupled to the first stimulation element 103. In that regard, the one or more temperature sensors 214 may be configured to sense the temperature of the first stimulation element 103. In that manner, in addition to modification through the user interface 108, electrical power being supplied to the first stimulation element 103 may also be modified in correlation with the sensed temperature. For example, in either of the cases of the first stimulation element 103 being the LED, the laser, the heating element, the cooling element, the vibration element, the electrode, or any combination thereof, the temperature of the first stimulation element 103 may be kept within the tolerance limit of the skin of the user by regulating the amount of electrical power being supplied to the first stimulation element 103. In several embodiments of the invention, as a safety feature, the sensed temperature based regulation of the first stimulation element 103 may override the user interface 108 based control of the first stimulation element 103. Furthermore, the first stimulation element 103 is located over a second compressible element 216. The second compressible element 216 may be embodied as a disc made from a deformable material such as silicone or other elastomeric materials.

Furthermore, a pressure sensor mesh 220 containing one or more pressure sensors is located below the second compressible element 216. When held sideways, the pressure sensor mesh 220 may be located towards the left side or the right side of the second compressible element 216. In that regard, terminology such as “a first side” and “a second side opposite to the first side” has been used in the claims to indicate the relative locations of elements of the device 100 with respect to each other. A plurality of touch switches 218 are located between the second compressible element 216 and the pressure sensor mesh 220. The one or more pressure sensors are configured to sense a pressure exerted on the second compressible element 216 for activation or deactivation of the first stimulation element 103 and a plurality of second stimulation elements 223 (elucidated in the following discussion). For example, when the user presses the first structure 102 onto the skin of the user, once a predefined pressure threshold is exceeded, the first stimulation element 103 and/or the plurality of second stimulation elements 223 may automatically be activated. However, similar to the first stimulation element 103, the plurality of second stimulation elements 223 may also be independently activated or deactivated through the user interface 108. Furthermore, as the user moves the device 100 away from their skin, the drop in pressure is sensed by the one or more pressure sensors and once the sensed pressure recedes beyond the predefined pressure threshold, the first stimulation element 103 and/or the plurality of second stimulation elements 223 may automatically be deactivated. It is to be noted that the sensed pressure-based activation and deactivation and/or the sensed temperature based regulation of the first stimulation element 103 and/or the plurality of second stimulation elements 223 may be an additional feature to and independent of the activation, deactivation, and regulation of the first stimulation element 103 and/or the plurality of second stimulation elements 223 through the user interface 108.

However, in several embodiments of the invention, the sensed pressure based deactivation and/or the sensed temperature based regulation of the first stimulation element 103 and/or the plurality of second stimulation elements 223 may be a safety feature that may override the user interface 108 based activation, deactivation and regulation of the first stimulation element 103 and/or the plurality of second stimulation elements 223. Moreover, in several alternate embodiments of the invention, the activation of the first stimulation element 103 and/or the plurality of second stimulation elements 223 through the user interface 108 may act as a first trigger and the sensed pressure exceeding the predefined pressure threshold may act as a second trigger and only after the first and the second trigger conditions have been satisfied, the first stimulation element 103 and/or the plurality of second stimulation elements 223 may start functioning. While during deactivation, the first stimulation element 103 and/or the plurality of second stimulation elements 223 may be independently deactivated based any trigger received through any one or more of the one or more temperature sensors 214, the pressure sensor mesh 220, the user interface 108, and combinations thereof.

The first structure 102 further includes a Printed Circuit Board (PCB) 224. The data and power transfer port 107 and the user interface 108 are electrically coupled to the PCB 224. Further, the first stimulation element 103 is electrically coupled to the PCB 224 through the resistive heating coil 212. The plurality of second stimulation elements 223 may be surface mounted or soldered onto the PCB 224 and therefore are also electrically coupled to the PCB 224. In construction, the plurality of second stimulation elements 223 defines a periphery surrounding the first stimulation element 103. Such a construction would allow combinational therapy to be provided to the user. For example, the first stimulation element 103 may be in contact with the skin of the user and may provide heating, cooling, or electrode therapy to the user, while the plurality of second stimulation elements 223 may provide light therapy to the user by emitting electromagnetic radiations with wavelengths ranging between 300 nm and 1200 nm. The first stimulation element 103 and the plurality of second stimulation elements 223 at least partially constitute the one or more stimulation elements of the device 100. Additional stimulation elements may be provided in other locations in the first structure 102 or in predefined locations in the second structure 104. In several embodiments of the invention, the plurality of second stimulation elements 223 may include a plurality of irradiation sources such as LEDs and/or lasers configured to emit electromagnetic radiation with wavelength ranges of 300 nm to 1200 nm. In that regard, a diaphanous cover layer 222 is disposed over the plurality of second stimulation elements 223. In several alternate embodiments of the invention, the plurality of second stimulation elements 223 may not include LEDs or laser but may include the heating elements, the cooling elements, the vibration elements, and electrodes. In such alternate embodiments, the diaphanous cover layer 222 may not be included in the first structure 102.

The first structure 102 further includes an electrical power source 228 electrically coupled to the PCB 224. In several embodiments of the invention, the electrical power source 228 includes a rectifier and a filter circuit configured to convert AC power received through the data and power transfer port 107 to DC power. In several alternate embodiments, the electrical power source 228 may include rechargeable batteries configured to be recharged by AC or DC electrical power supply received through the data and power transfer port 107. The rechargeable batteries may include Lithium-ion batteries, Lithium-polymer batteries, Nickel-Metal-Hydride batteries, etc. Alternately, the rechargeable batteries may be configured to be charged through a receiver induction coil 225 configured to receive power from a time-varying magnetic field generated by a transmitter induction coil (not shown) of a wireless charging device (not shown). The first structure 102 further includes a supporting bracket 230 such that the PCB 224 is located on a first side of the supporting bracket 230 and a lid 242 included in the second structure 104 is located on a second side of the bracket. The second side is opposite to the first side.

The one or more first compressible elements 106 are disposed between the supporting bracket 230 and the lid 242. Moreover, the one or more first compressible elements 106 are disposed on the second side of the supporting bracket 230. The device 100 further includes an expansion switch 232 coupled to the one or more first compressible elements 106 through a flexible diaphragm 236. As any one of the one or more first compressible elements 106 expands, the flexible diaphragm 236 also expands thereby activating the expansion switch 232. Alternately, when all of the one or more first compressible elements 106 are in the compressed state, the flexible diaphragm 236 also is fully compressed causing the expansion switch 232 to be deactivated. When activated, the expansion switch 232 is configured to activate the one or more stimulation elements and when deactivated, the expansion switch 232 is configured to deactivate the one or more stimulation elements. Furthermore, at least one of the one or more first compressible elements 106 includes an air transfer port 237. The air transfer port 237 is configured to receive air when a respective first compressible element, of the one or more first compressible elements 106, transitions from the compressed state to the expanded state, and to release air when the respective first compressible element transitions from the expanded state to the compressed state. In addition to the lid 242, the second structure 104 also includes a second fastening arrangement 240. In several embodiments of the invention, the second fastening arrangement 240 may be selected from a group consisting of suction cups, snap-fit arrangements, circular strips with adhesive agents, magnetic rings, etc. In that regard, one or both of the first structure 102 and the second structure 104 may include a respective fastening arrangement (the first fastening arrangement 202 for the first structure 102 and the second fastening arrangement 240 for the second structure 104) configured to mechanically couple the first structure or the second structure to another object.

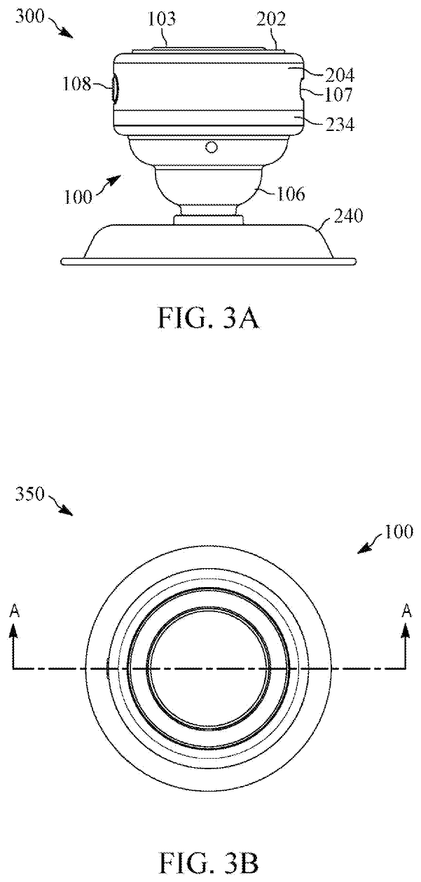

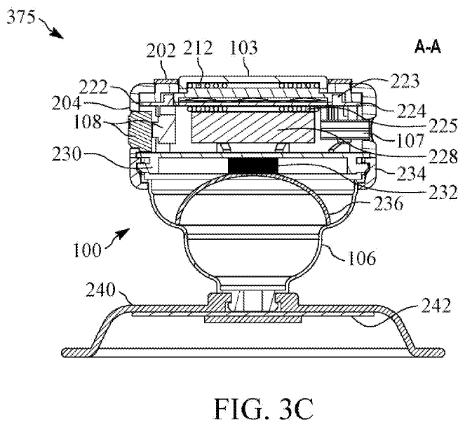

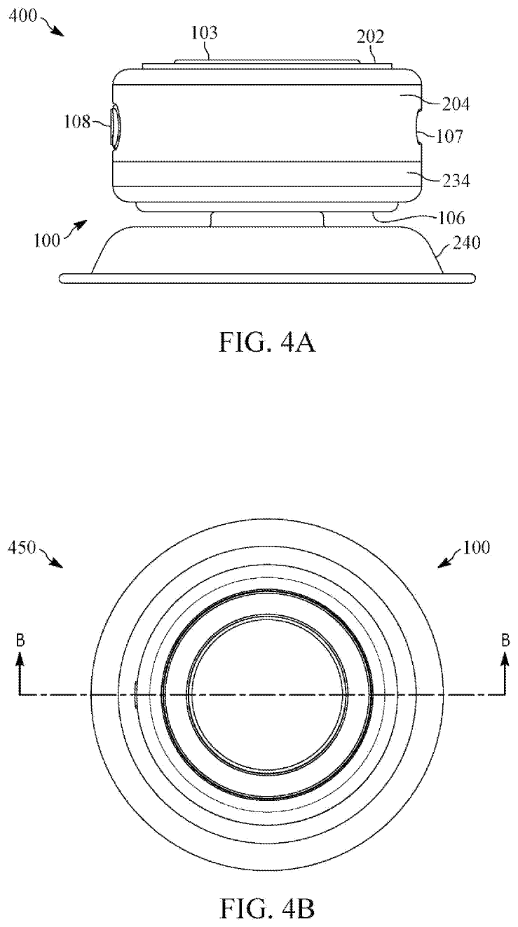

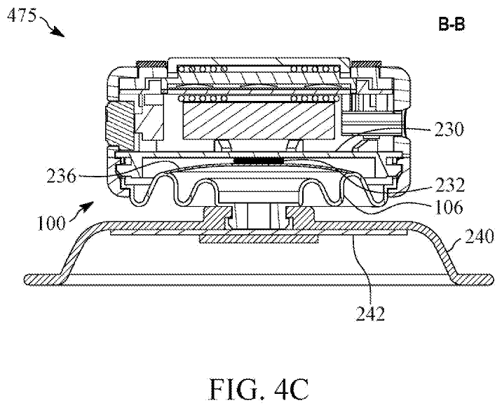

FIG. 3A illustrates a front view 300 of the therapeutic device 100 in an open condition, in accordance with an embodiment of the present invention. FIG. 3B illustrates a bottom view 350 of the therapeutic device 100 of FIG. 3A. FIG. 3C illustrates a front sectional view 375 of the therapeutic device 100 of FIGS. 3A and 3B, sectioned along a plane A-A of FIG. 3B. In the open condition, the one or more first compressible elements 106 are in the expanded state. FIG. 4A illustrates a front view 400 of the therapeutic device 100 in a closed condition, in accordance with an embodiment of the present invention. FIG. 4B illustrates a bottom view 450 of the therapeutic device 100 of FIG. 4A. FIG. 4C illustrates a front sectional view 475 of the therapeutic device 100 of FIGS. 4A and 4B, sectioned along a plane B-B of FIG. 4B. In the closed condition, the one or more first compressible elements 106 are in the compressed state.

FIG. 5 illustrates the therapeutic device 100 in communication with a user computing device 520, in accordance with an embodiment 500 of the present invention. The therapeutic device 100 is envisaged to include a control module 502. The control module 502 is envisaged to include a processor 504, a memory unit 506, a storage device 508, and a communication interface 510. The communication interface 510 is configured to enable communication with the user computing device 520 through a communication network 512. The user computing device 520 may be one or more of a smartphone, a Personal Digital Assistant (PDA), a notebook PC, a tablet PC, a desktop PC, a dedicated remote control, etc. The memory unit 506, in that regard, may include machine-readable instructions, that when executed by the processor 504 may enable the processor 504 to receive a control input signal from the user computing device 520. The processor 504 may further be enabled to modify the operational characteristics of the first stimulation element 103 and the plurality of second stimulation elements 223 in response to the receipt of the control input signal. The operational characteristics may vary from one kind of stimulation element to another kind of stimulation element. For example, the intensity, wavelength, or pulse width of the electromagnetic radiation emitted by the LEDs or laser may be modified, maximum or minimum surface temperatures of the heating element and cooling element may be modified, frequency and amplitude of vibration of the vibratory element may be modified, electrical potential or current value of the electrodes may be modified, etc.

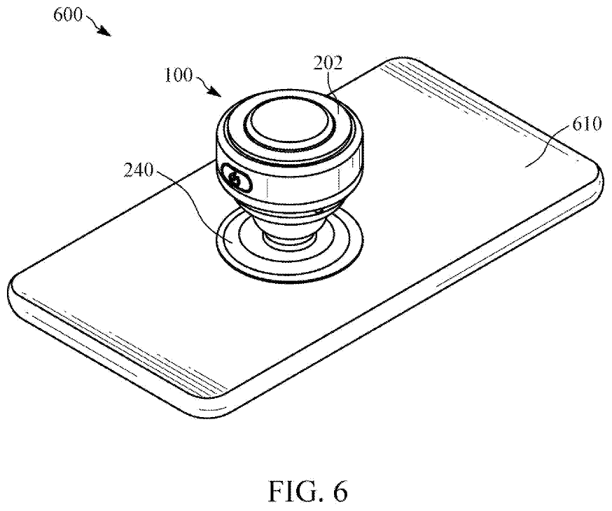

FIG. 6 illustrates the therapeutic device 100 mechanically coupled to another electronic device 610, in accordance with an embodiment 600 of the present invention. The therapeutic device 100 is mechanically coupled to the other electronic device 610 through the second fastening arrangement 240. In that regard, the second fastening arrangement 240 may be a suction cup, a magnetic strip, or a circular strip with an adhesive agent. It is to be noted here that the therapeutic device 100 may also be attached to the other electronic device 610, in the top portion, through the first fastening arrangement 202.

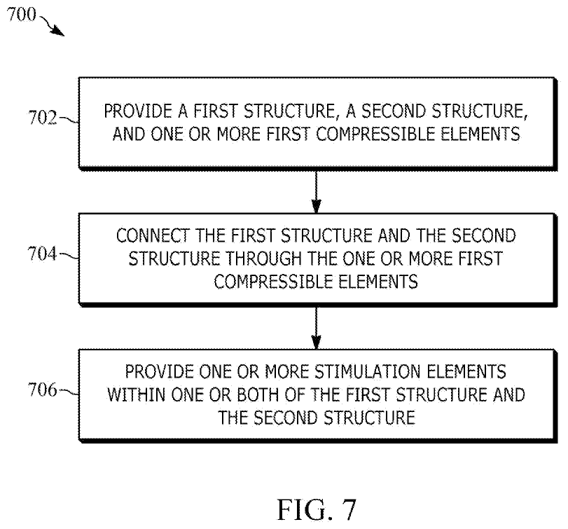

FIG. 7 illustrates a flowchart depicting a method 700 of manufacturing the therapeutic device 100, in accordance with an embodiment of the present invention. The method 700 begins at Step 702. At Step 702, the first structure 102, the second structure 104, and the one or more first compressible elements 106 are provided. The one or more first compressible elements 106 are configured to transition between the compressed state and the expanded state. At Step 704, the first structure 102 and the second structure 104 are connected through the one or more first compressible elements 106. At Step 706, the one or more stimulation elements are provided in one or both of the first structure 102 and the second structure 104.

The embodiments of the present invention as presented above offer several advantages. For instance, the therapeutic device 100 is compact in construction and can be further collapsed for convenient storage and handling. The therapeutic device 100 can be used for providing several different combinations of distinct therapies such as heating, cooling, irradiation, etc. with a very small footprint in terms of power and space requirements. The design is simple in construction and does not require manufacturing of complex geometries. Moreover, the therapeutic device 100 can be attached to several other objects allowing the therapeutic device 100 to be provided a further handle or be used as a handle for the other objects.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapeutic device, comprising:
a first structure and a second structure connected through one or more first compressible elements, one or both of the first structure and the second structure comprising one or more stimulation elements,
wherein the one or more first compressible elements are configured to transition between a compressed state and an expanded state.

2. The therapeutic device as claimed in claim 1, wherein the one or more stimulation elements are selected from a group consisting of Light Emitting Diodes (LEDs), lasers, heating elements, cooling elements, vibration elements, electrodes and combinations thereof.

3. The therapeutic device as claimed in claim 1, wherein:
the first structure comprises:
a supporting bracket,
a Printed Circuit Board (PCB) located on a first side of the supporting bracket, and
a first stimulation element, a plurality of second stimulation elements, and an electrical power source electrically coupled to the PCB, the first stimulation element and the plurality of second stimulation elements at least partially constituting the one or more stimulation elements, and the second structure comprises:
a lid located on a second side of the supporting bracket, the second side being opposite to the first side,
wherein the one or more first compressible elements are disposed between the supporting bracket surface and the lid, on the second side of the supporting bracket.

4. The therapeutic device as claimed in claim 3, further comprising one or more pressure sensors electrically coupled to the PCB, a second compressible element in contact the one or more pressure sensors towards the second side, wherein the one or more pressure sensors are configured to sense a pressure exerted on the second compressible element for activation and deactivation of the one or more of the first stimulation element and the plurality of second stimulation elements.

5. The therapeutic device as claimed in claim 4, wherein the first stimulation element is a heating plate located over the second compressible element, the heating plate thermally coupled to a resistive heating coil, one or more temperature sensors thermally coupled to the first stimulation element, the one more temperature sensors configured to sense a temperature of the first stimulation element, such that, in use, electrical power being supplied to the heating element is modified in correlation with the sensed temperature.

6. The therapeutic device as claimed in claim 3, wherein, in construction, the plurality of second stimulation elements defines a periphery surrounding the first stimulation element.

7. The therapeutic device as claimed in claim 3, wherein the plurality of second elements comprises Light Emitting Diodes (LEDs) and/or lasers, and the first structure further comprises a diaphanous cover layer disposed over the plurality of second stimulation elements, towards the first side.

8. The therapeutic device as claimed in claim 1, further comprising an expansion switch coupled to the one or more first compressible elements, the expansion switch configured to activate the one or more stimulation elements when at least one of the one or more first compressible elements are in the expanded state and deactivate the one or more stimulation elements when each one of the one or more compressible elements are in the compressed state.

9. The therapeutic device as claimed in claim 1, wherein the one or more stimulation elements comprise a plurality of irradiation sources configured to emit electromagnetic radiation with wavelength ranges of 300 nm to 1200 nm.

10. The therapeutic device as claimed in claim 1, wherein at least one of the one or more first compressible elements comprises an air transfer port configured to receive air when a respective first compressible element, of the one or more first compressible elements, transitions from the compressed state to the expanded state, and to release air when the respective first compressible element transitions from the expanded state to the compressed state.

11. The therapeutic device as claimed in claim 1, further comprising a receiver induction coil electrically coupled to the electrical power source, the receiver induction coil configured to receive electrical power from a time-varying magnetic field generated by a transmitter induction coil of a wireless charging device.

12. The therapeutic device as claimed in claim 1, further comprising a communication interface configured to receive a control input signal from a user computing device, such that, in use, operational characteristics of one or more of the first stimulation element and the plurality of second stimulation elements are modified in response to the receipt of the control input signal.

13. The therapeutic device as claimed in claim 1, wherein one or both of the first structure and the second structure comprise a respective fastening arrangement configured to mechanically couple the first structure or the second structure to another object.

14. A therapeutic device, comprising:
a first structure and a second structure connected through one or more first compressible elements, one or both of the first structure and the second structure comprising one or more stimulation elements,
wherein the one or more first compressible elements are configured to transition between a compressed state and an expanded state,
wherein the one or more stimulation elements are at least partially constituted by a first stimulation element and a plurality of second stimulation elements, and
wherein, in construction, the plurality of second stimulation elements defines a periphery surrounding the first stimulation element.

15. The therapeutic device as claimed in claim 14, wherein:
the first structure comprises:
a supporting bracket,
a Printed Circuit Board (PCB) located on a first side of the supporting bracket, the plurality of second stimulation elements, the first stimulation element, and an electrical power source electrically coupled to the PCB, and the second structure comprises:
a lid located on a second side of the supporting bracket, the second side being opposite to the first side,
wherein the one or more first compressible elements are disposed between the supporting bracket surface and the lid, on the second side of the supporting bracket.

16. The therapeutic device as claimed in claim 15, further comprising one or more pressure sensors electrically coupled to the PCB, a second compressible element in contact the one or more pressure sensors towards the second side, wherein the one or more pressure sensors are configured to sense a pressure exerted on the second compressible element for activation and deactivation of the one or more of the first stimulation element and the plurality of second stimulation elements.

17. The therapeutic device as claimed in claim 16, wherein the first stimulation element is a heating plate located over the second compressible element, the heating plate thermally coupled to a resistive heating coil, one or more temperature sensors thermally coupled to the first stimulation element, the one more temperature sensors configured to sense a temperature of the first stimulation element, such that, in use, electrical power being supplied to the heating element is modified in correlation with the sensed temperature.

18. The therapeutic device as claimed in claim 15, wherein the plurality of second elements comprises Light Emitting Diodes (LEDs) and/or lasers, and the first structure further comprises a diaphanous cover layer disposed over the plurality of second stimulation elements, towards the first side.

19. The therapeutic device as claimed in claim 14, further comprising an expansion switch coupled to the one or more first compressible elements, the expansion switch configured to activate the one or more stimulation elements when at least one of the one or more first compressible elements are in the expanded state and deactivate the one or more stimulation elements when each one of the one or more compressible elements are in the compressed state.

20. A method of manufacturing a therapeutic device, the method comprising:
providing a first structure, a second structure, and one or more first compressible elements;
connecting the first structure and the second structure through the one or more first compressible elements; and
providing one or more stimulation elements within one or both of the first structure and the second structure,
wherein the one or more first compressible elements are configured to transition between a compressed state and an expanded state.
